SEQUENCE LISTING (10 sequences)

SEQ ID NO: 1 - DNA, 49 bp, synthetic construct
  source 1..49: mol_type = other DNA; note = DDX58 loop 48 nt v1
ggctgtccactgtagagttgctgtatgtagcaactctacagtggtcagc

SEQ ID NO: 2 - DNA, 49 bp, synthetic construct
  source 1..49: mol_type = other DNA; note = DDX58 loop 48 nt v2
ggctgtccacggcagagttgctgtatgtagcaactctgcggtggtcagc

SEQ ID NO: 3 - DNA, 45 bp, synthetic construct
  source 1..45: mol_type = other DNA; note = GluR loop v1
ggtgaatagtataacaatatgctaaatgttgttatagtatccacc

SEQ ID NO: 4 - DNA, 55 bp, synthetic construct
  source 1..55: mol_type = other DNA; note = GluR loop v9.4
ggtgtcgagaagaggagaacaatatgctaaatgttgttctcgtctcctcgacacc

SEQ ID NO: 5 - DNA, 26 bp, synthetic construct
  source 1..26: mol_type = other DNA; note = histone 3-UTR mutated stem-loop (SLmt)
ccaaaggctcttctcagagccaccca

SEQ ID NO: 6 - DNA, 26 bp, synthetic construct
  source 1..26: mol_type = other DNA; note = histone 3-UTR stem-loop (SL)
ccaaaggctcttttcagagccaccca

SEQ ID NO: 7 - DNA, 37 bp, synthetic construct
  source 1..37: mol_type = other DNA; note = truncated and modified version of prequeosine1-1 riboswitch aptamer (teQ1)
cgcggttctatctagttacgcgttaaaccaactagaa

SEQ ID NO: 8 - DNA, 51 bp, synthetic construct
  source 1..51: mol_type = other DNA; note = frameshifting pseudoknot from Moloney murine leukemia virus (tmp)
gggtcaggagccccccccctgaacccaggataaccctcaaagtcggggggc

SEQ ID NO: 9 - RNA, 24 nt, synthetic construct
  source 1..24: mol_type = other RNA; note = Forward primer
aacttcagcctgctcaaacaagcc

SEQ ID NO: 10 - RNA, 20 nt, synthetic construct
  source 1..20: mol_type = other RNA; note = Reverse primer
cagccctggtcttgtagttg

(19) United States
(12) Patent Application Publication
(10) Pub. No.: US 2026/0218188 A1
(43) Pub. Date: Jul. 30, 2026

(54) ANTISENSE OLIGONUCLEOTIDES FOR RNA EDITING AND METHODS FOR USING THE SAME

(71) Applicant: Deutsches Krebsforschungszentrum Stiftung des öffentlichen Rechts, Heidelberg (DE)

(72) Inventors: Riccardo PECORI, Heidelberg (DE); Annette ARNOLD, Heidelberg (DE)

(73) Assignee: Deutsches Krebsforschungszentrum Stiftung des öffentlichen Rechts, Heidelberg (DE)

(21) Appl. No.: 19/149,086

(22) PCT Filed: Jan. 17, 2024
(86) PCT No.: PCT/EP2024/051033

§ 371 Date: Jul. 17, 2025

(30) Foreign Application Priority Data
Jan. 18, 2023 (EP) 23152263.2

(51) Int. Cl. C12N 15/113

(52) U.S. Cl. CPC C12N 15/113; C12N 2310/11; C12N 2310/531

(57) ABSTRACT

The present invention concerns antisense oligonucleotides and methods for using the same. In particular, it relates to an antisense oligonucleotide having the following elements: 3′-A1-B-A2-D-A3-5′, wherein A1 is a nucleotide sequence having a length of from 6 to 16 nucleotides; B is the nucleotide cytosine; A2 is a nucleotide sequence having a length of from 33 to 37 nucleotides; D is an internal loop-forming nucleotide sequence having a length of 4 nucleotides; and A3 is a nucleotide sequence having a length of from 10 to 20 nucleotides, wherein elements A1, A2 and A3 are contiguous nucleotide sequences that are complementary to a target nucleotide sequence, and wherein the cytosine of element B forms a base-pair mismatch with adenosine in the target nucleotide sequence upon hybridization of elements A1, A2 and A3 to the target nucleotide sequence. Furthermore, the present invention relates to an expression cassette comprising a promoter sequence driving the expression of a nucleotide encoding said antisense oligonucleotide. In addition, the present invention concerns a vector comprising a nucleic acid encoding said antisense oligonucleotide or at least said one expression cassette. The present invention further envisages use of said antisense oligonucleotide, said expression cassette or the vector for editing RNA in a cultured cell or in vitro, as well as a method for editing RNA comprising contacting the RNA to be edited in cultured cell or in vitro. Moreover, the present invention relates to a pharmaceutical composition comprising the antisense oligonucleotide, the expression cassette or the vector. Further encompassed is an antisense, an expression cassette or a vector for use in treating and/or preventing a disease or disorder associated with mutated RNA. The present invention also refers to a method for treating and/or preventing a disease associated with mutated RNA.

The present invention concerns antisense oligonucleotides and methods for using the same. In particular, it relates to an antisense oligonucleotide having the following elements: 3′-A₁-B -A₂-D-A₃-5′, wherein A₁ is a nucleotide sequence having a length of from 6 to 16 nucleotides; B is the nucleotide cytosine; A₂ is a nucleotide sequence having a length of from 33 to 37 nucleotides; D is an internal loop-forming nucleotide sequence having a length of 4 nucleotides; and A₃ is a nucleotide sequence having a length of from 10 to 20 nucleotides, wherein elements A₁, A₂ and A₃ are contiguous nucleotide sequences that are complementary to a target nucleotide sequence, and wherein the cytosine of element B forms a base-pair mismatch with adenosine in the target nucleotide sequence upon hybridization of elements A1, A₂ and A₃ to the target nucleotide sequence. Furthermore, the present invention relates to an expression cassette comprising a promoter sequence driving the expression of a nucleotide encoding said antisense oligonucleotide. In addition, the present invention concerns a vector comprising a nucleic acid encoding said antisense oligonucleotide or at least said one expression cassette. The present invention further envisages use of said antisense oligonucleotide, said expression cassette or the vector for editing RNA in a cultured cell or in vitro, as well as a method for editing RNA comprising contacting the RNA to be edited in cultured cell or in vitro. Moreover, the present invention relates to a pharmaceutical composition comprising the antisense oligonucleotide, the expression cassette or the vector. Further encompassed is an antisense, an expression cassette or a vector for use in treating and/or preventing a disease or disorder associated with mutated RNA. The present invention also refers to a method for treating and/or preventing a disease associated with mutated RNA.

Recent studies have demonstrated that RNA undergoes various modifications in a manner similar to DNA. These modifications affect function and stability of RNA and, thus, play an important role in many cellular and biological processes, such as transcription, pre-mRNA splicing, RNA export, mRNA translation and RNA degradation. Among these modifications, RNA editing is unique since it not only can alter the cellular fate of RNA molecules but also alters their sequence relative to the genome in a site-specific and precise manner, which leads to a considerable increase of the repertoire of genome-encoded RNAs (WO 2022/091100 A1). Examples of RNA editing are adenosine to inosine (A-to-I) or cytidine to uridine (C-to-U) conversions through enzymes called adenosine deaminase and cytidine deaminase, respectively.

The most studied form of RNA editing system in Metazoans is the adenosine deaminase acting on RNA (ADAR) gene family catalyse deamination of adenosine nucleotides to inosines. ADAR is a multi-domain protein comprising a recognition domain and a catalytic domain. The recognition domain recognizes a specific double-stranded RNA sequence and/or conformation, whereas the catalytic domain converts an adenosine into inosine in the target RNA by deamination of the nucleobase. Inosine is read as guanine by the translational machinery of the cell, i.e. if an edited adenosine is in a coding region of an mRNA or pre-mRNA, it can recode the protein sequence. RNA editing occurs primarily within noncoding regions, and only a small percentage takes place in coding regions resulting in amino acid change. For example, A-to-I editing occurs in humans mostly in introns and untranslated regions (UTRs) of protein coding genes.

Unlike DNA editing, RNA editing is reversible. Hence, the interest in RNA editing increased significantly due to the considerable therapeutic potential of nucleic acid editing as it provides a safer option than DNA editing or gene therapy since potential adverse effects and off-target edits should be reversible and dose-dependent. A platform based on RNA editing could have broad utility as a therapeutic intervention and potential cure for a wide range of diseases caused by genetic point mutations and other genetic lesions. In particular, RNA editing strategies based on ADAR would be preferable in a therapeutic setting due to its ubiquitous nature and since ectopic expression of an engineered protein could be replaced with administration of an oligonucleotide drug.

Various study groups demonstrated remarkable success in RNA editing. For instance, Merkle et al. (2019) described the engineering of chemically optimized antisense oligonucleotides that recruit endogenous human ADARs to edit endogenous transcripts and demonstrated repair of the clinically relevant PiZZ mutation, which causes al-antitrypsin deficiency. Qu et al. (2019) described engineered ADAR-recruiting RNAs to recruit native ADAR enzymes to alter specific adenosines to inosines with increased efficiency.

However, despite considerable progress in studying the mechanisms ADAR, the fundamental rules determining the exact location of editing and steering ADAR to said location, as wells as the extent of editing, remain poorly understood (Uzonyi et al., 2021). In addition, the low specific delivery, low editing efficiency, high bystander off-target editing and high production costs continue to be unsolved obstacles.

Thus, there is a need for nucleic acid molecules for highly specific and efficient RNA editing of a target RNA sequence.

The technical problem underlying the present invention may be seen as the provision of means and methods for complying with the aforementioned need. The technical problem is solved by the embodiments characterized in the claims and herein below.

The present invention relates to an antisense oligonucleotide having the following elements:

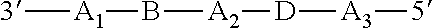

wherein
A₁ is a nucleotide sequence having a length of from 6 to 16 nucleotides;
B is the nucleotide cytosine;
A₂ is a nucleotide sequence having a length of from 33 to 37 nucleotides;
D is an internal loop-forming nucleotide sequence having a length of 4 nucleotides; and
A₃ is a nucleotide sequence having a length of from 10 to 20 nucleotides,
wherein elements A₁, A₂ and A₃ are contiguous nucleotide sequences that are complementary to a target nucleotide sequence, and wherein the cytosine of element B forms a base-pair mismatch with adenosine in the target nucleotide sequence upon hybridization of elements A1, A₂ and A₃ to the target nucleotide sequence.

It is to be understood that in the specification and in the claims, “a” or “an” can mean one or more of the items referred to in the following depending upon the context in which it is used. Thus, for example, reference to “an” item can mean that at least one item can be utilized.

As used in the following, the terms “have”, “comprise”, or “include” are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

The terms “particularly”, “more particularly”, “typically”, and “more typically” or similar terms are used in conjunction with additional and/or alternative features, without restricting alternative possibilities. Thus, features introduced by these terms are additional and/or alternative features and are not intended to restrict the scope of the claims in any way. The invention may, as the skilled person will recognize, be performed by using further alternative features. Similarly, features introduced by “in an embodiment of the invention” or similar expressions are intended to be additional and/or alternative features, without any restriction regarding alternative embodiments of the invention, without any restrictions regarding the scope of the invention and without any restriction regarding the possibility of combining the features introduced in such way with other additional and/or alternative or non-additional and/or alternative features of the invention.

Further, as used in the following, the terms “preferably”, “more preferably”, “most preferably”, “particularly”, “more particularly”, “typically”, and “more typically” are used in conjunction with features in order to indicate that these features are preferred features, i.e. the terms shall indicate that alternative features may also be envisaged in accordance with the invention.

Further, it will be understood that the term “at least one” as used herein means that one or more of the items referred to following the term may be used in accordance with the invention. For example, if the term indicates that at least one item shall be used this may be understood as one item or more than one item, i.e. two, three, four, five, or any other number. Depending on the item the term refers to, the skilled person understands as to what upper limit the term may refer, if any.

The term “about” as used herein means that with respect to any number recited after said term an interval accuracy exists within in which a technical effect can be achieved. Accordingly, the term “about” in the context of the present invention means ±20%, ±10%, ±5%, ±2%, or ±1% from the indicated parameters or values. This also takes into account usual deviations caused by measurement techniques and the like.

The term “oligonucleotide” is generally understood by the skilled person as a molecule comprising two or more covalently linked nucleosides. Such covalently bound nucleosides may also be referred to as nucleic acid molecules or oligomers. The oligonucleotides referred to in the present invention may be therapeutic oligonucleotides below 200 nucleotides in length. Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides comprise a ribose sugar moiety, a nucleobase moiety and one or more phosphate groups, which is absent in nucleosides.

The term “nucleobase” includes the purine (e.g. adenine and guanine) and pyrimidine (e.g. uracil, thymine and cytosine) moiety present in nucleosides and nucleotides, which form hydrogen bonds in nucleic acid hybridization.

The term “nucleobase” refers both to naturally occurring nucleobases such as adenine, guanine, cytosine, thymidine, uracil, xanthine and hypoxanthine, as well as non-naturally occurring variants. Such variants are for example described in Hirao et al. (2012). The terms “adenine”, “guanine”, “cytosine”, “thymine”, “uracil” and “hypoxanthine” shall refer to the nucleobases as such. The terms “adenosine”, “guanosine”, “cytidine”, “thymidine”, “uridine” and “inosine” shall refer to the nucleobases linked to the ribosyl sugar. The nucleobase moieties may be indicated by the letter code for each corresponding nucleobase, e.g. A, T, G, C, U or I, wherein each letter may optionally include modified nucleobases of equivalent function. The nucleobase moiety can be modified by changing the purine or pyrimidine into a modified purine or pyrimidine, such as substituted purine or substituted pyrimidine, e.g. as a nucleobase selected from isocytosine, pseudoisocytosine, 5-methyl cytosine, 5-thiozolo-cytosine, 5-propynyl-cytosine, 5-propynyl-uracil, 5-bromouracil, 5-thiazolo-uracil, 2-thio-uracil, 2-thio thymine, inosine, diaminopurine, 6-aminopurine, 2-aminopurine, 2,6-diaminopurine and 2-chloro-6-aminopurine.

An “antisense oligonucleotide” (ASO) as used herein, refers to a single strand DNA and/or RNA molecule that is capable of interfering with DNA and/or RNA processing. Antisense oligonucleotides comprise a nucleic acid sequence, which is complementary to a specific RNA or DNA sequence.

The term “complementary” describes the capacity for Watson-Crick base-pairing of nucleosides/nucleotides. Watson-Crick base-pairs are guanine (G)-cytosine (C) and adenine (A)-thymine (T)/uracil (U). Further, inosine (I) pairs with cytosine (C) in a Watson-Crick bonding configuration. It will be understood that oligonucleotides may comprise nucleosides with modified nucleobases, for example 5-methyl cytosine is often used in place of cytosine. As such, the term complementarity encompasses Watson-Crick base-pairing between non-modified and modified nucleobases.

The percentage of complementarity (“% complementary”), refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity). Further, inosine is considered to be identical to a guanine for the purpose of calculating % identity. Sequence identity can be determined using any protein or nucleic acid sequence alignment algorithm such as Blast, ClustalW and MUSCLE.

Typically, an antisense oligonucleotide will bind, in a sequence-specific manner, to its respective complementary oligonucleotides, DNA, or RNA, thereby interfering with DNA and/or RNA processing. It is known to those skilled in the art that antisense oligonucleotides may interfere with mRNA processing through RNase H-mediated degradation, translational arrest, modulation of splicing or they may act through steric hindrance of proteins. Means and methods for the design and synthesis of antisense oligonucleotides are well-known in the art and include, for example, rational design, chemical modifications and design of antisense oligonucleotides containing locked nucleic acids (LNA) as well as solid-phase chemical synthesis. Antisense oligonucleotides can be chemically synthesized or expressed within the cell, for example, by introduction of respective recombinant DNA construct. It will be understood by those skilled in the art that such a DNA construct may contain, in addition to a nucleic acid sequence encoding he antisense oligonucleotide, regulatory elements such as an enhancer, a constitutive or inducible promoter or a terminator as described elsewhere herein in more detail. Preferably, the antisense oligonucleotide has a total length of at least 54, at least 56, at least 58, at least 60, at least 62, at least 64, at least 66, at least 68, at least 70, at least 72, at least 74, at least 76, at least 78 or more nucleotides. The antisense oligonucleotide may comprise deoxyribonucleotides, ribonucleotides, or a combination of both.

The antisense oligonucleotide of the present invention comprises the elements A₁, A₂ and A₃. Element A₁ may have a length from 6 to 16 nucleotides, i.e. it may have a length of 6 nucleotides, 7 nucleotides, 8 nucleotides, 9 nucleotides, 10 nucleotides, 11 nucleotides, 12 nucleotides, 13 nucleotides, 14 nucleotides, 15 nucleotides or 16 nucleotides. Element A₂ may have a length from 33 to 37 nucleotides, i.e. it may have a length of 33 nucleotides, 34 nucleotides, 35 nucleotides, 36 nucleotides or 37 nucleotides. Element A₃ may have a length from 10 to 20 nucleotides, i.e. it may have a length of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides or 20 nucleotides. Preferably, elements A₁, A₂ and A₃ are contiguous nucleotide sequences that are complementary to a target nucleotide sequence, i.e. a nucleic acid sequence that can form a double-stranded structure by matching base pairs to another nucleic acid sequence. The target nucleotide sequence is typically part of an RNA encoded by a gene of interest. The target RNA may, thus, be an mRNA that will be translated into a protein or a regulatory RNA. The length and the % complementarity may be routinely determined by a skilled person. For example, the antisense oligonucleotide or contiguous nucleotide sequence can be at least 90% complementary, at least 91% complementary, at least 92% complementary, at least 93% complementary, at least 94% complementary, at least 95% complementary, at least 96% complementary, at least 97% complementary, at least 98% complementary or at least 99% complementary to a target nucleotide sequence. In some examples, the contiguous nucleotide sequence is fully complementary to the target nucleotide sequence.

However, the present invention envisages that the antisense oligonucleotide sequence comprises less than 100% complementarity to the target nucleotide sequence. Specifically, it is envisaged that the antisense oligonucleotides of the present invention may comprise at least one base-pair mismatch to the target nucleotide sequence. As used herein, the term “base-pair mismatch” refers to at least one nucleotide having no base pair complementation with a given nucleotide, for example G-A, G-U, C-A, U-C, A-A, G-G, C-C, or U-U. Preferably, the mismatch (element B) is located downstream of element A₁ and upstream of element A₂ of the antisense oligonucleotide. More preferably, the base-pair mismatch (represented by element B in the antisense oligonucleotide) is formed between a cytosine of the antisense oligonucleotide and an adenosine of the target nucleotide sequence. A mismatch results in a disruption of the double-stranded structure formed upon hybridization of the antisense oligonucleotide with its target. The term “hybridization” as used herein, refers to a reaction in which the nucleotide sequence, i.e. the elements A₁, A₂ and A₃ of the antisense oligonucleotide, and the target nucleotide sequence react to form a complex that is stabilized via non-covalent bonding between the bases of the nucleotide residues. The non-covalent bonding may occur by e.g. Watson Crick base pairing, Hoogstein binding or in any other sequence specific manner, including non-naturally occurring/synthetic nucleotides or bonds between them.

Examples of structural disruptions include, but are not limited to a bulge, an internal loop, a stem loop (hairpin loop), a junction or a combination thereof. Preferably, the structural disruption is an internal loop. In certain examples, the internal loop may also be formed in the target nucleotide sequence. Preferably, the nucleotide sequence and the complementary target nucleotide sequence form double-stranded structures both upstream and downstream of the disruption.

Internal loops are unpaired stretches of nucleotides located within one strand of a nucleic acid duplex which is formed by hydrogen-bonded bases including canonical Watson-Crick and non-canonical base pairs. Internal loop sizes can vary from a single unpaired residue up to several nucleotides that form frequently flexible extrusions from pseudo-continuous double helices. Internal loops (interior loops) occur in RNA at locations where the double stranded RNA separates due to lacking Watson-Crick base pairing between the nucleotides. Typically, internal loops occur in middle of a stretch of double stranded RNA and can form symmetrical or asymmetrical structures, e.g. C-loop, docking-elbow, k-turn, right-angle, sarcin/ricin loop, twist-up motif, UAA/GAN internal loop motif. A stem loop occurs when two regions of the same RNA strand base-pair to form a double helix that ends in an unpaired loop. Stem loops consist of a stem, double helix and a loop which links the stem. Typically the length of the loop is between 3 to 8 nucleotides. RNA junctions are constructs where two or more stems meet and usually contain unmatched bases.

The antisense oligonucleotide of the present invention comprises an element D, which is an internal loop-forming nucleotide sequence having a length of 4 nucleotides. It is to be understood that the internal loop-forming nucleotide sequence is a base-pair mismatch with a length of 4 nucleotides, and thus, forming a disruption with a length of 4 nucleotides. Preferably, the internal loop is formed in the nucleotide sequence of the antisense oligonucleotide. In certain examples, the internal loop may also be formed in the target nucleotide sequence. Preferably, the mismatch (element D) is located downstream of element A₂ and upstream of element A₃ of the antisense oligonucleotide.

In one embodiment, the antisense oligonucleotide may be or comprises a single-stranded antisense oligonucleotide. Preferably, the single-stranded antisense oligonucleotide is a single-stranded antisense RNA oligonucleotide. Oligonucleotide molecules are commonly produced in the laboratory by solid-phase chemical synthesis followed by purification and isolation. The antisense oligonucleotide may also be delivered to cells using a vector, such as viral vectors (e.g. lentiviral or adenoviral vectors from which they are then transcribed to produce the RNA antisense oligonucleotide).

When referring to a sequence of oligonucleotides, reference is made to the sequence or order of nucleobase moieties, or modifications thereof, of the covalently linked nucleotides or nucleosides. As such, the oligonucleotide of the invention may comprise one or more modified nucleosides or nucleotides.

The present invention envisages RNA editing by using the enzyme Adenosine Deaminase Acting on RNA (ADAR). The term “ADAR” refers to a double-stranded RNA specific adenosine deaminase, which catalyses the hydrolytic deamination of adenosine to inosine in double-stranded RNA (dsRNA), also referred to as A to I editing. Typically, ADARs share a common modular organization, which consists of a variable N-terminal region, a double-stranded RNA binding domain and a zinc containing catalytic domain. Preferably, the ADAR is of human origin. The ADAR may be ADAR1, ADAR2 or ADAR3.

ADAR1, ADAR2 and ADAR3 proteins referred to in accordance with the present invention are preferably of human origin having an amino acid sequence as deposited under UniProt accession number P55265, P52948 and Q9NS39, respectively. It will be understood that the terms “ADAR1”, “ADAR2” and “ADAR3 also relate to variants of said proteins. Such variants have at least the same essential biological and immunological properties as the aforementioned ADAR1, ADAR2 and ADAR3 protein. In particular, they share the same essential biological and immunological properties if they are detectable by the same specific assays referred to in this specification. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the specific amino acid sequence of ADAR1, ADAR2 or ADAR3 protein, preferably over the entire length of the said ADAR1, ADAR2 or ADAR3 proteins, respectively. It will be further understood that the ADAR shall be catalytically active. For example, the human ADAR1 and ADAR2 isoforms are known to be catalytically active.

The ADAR may be endogenous ADAR, recombinantly expressed in the target cell or delivered to the target cell.

ADAR may be steered to the antisense oligonucleotide sequence/target nucleic acid sequence complex by a specific nucleic acid sequence or secondary structure that is present on the ASO. Accordingly, in one embodiment the antisense oligonucleotide has a further element E following 5′ of element A₃, wherein element E is a recruitment domain for recruiting a deaminase. Preferably, the recruitment domain is for recruiting adenosine deaminase. More preferably, the recruitment domain is for recruiting adenosine deaminase acting on RNA (ADAR). Most preferably, the recruitment domain is for recruiting ADAR1 or ADAR2. The term “ADAR recruitment domain” refers to a nucleic acid sequence or structure that mediates binding of ADAR to the complementary nucleic acid sequence:target nucleic acid sequence complex in a direct or indirect manner. Exemplary ADAR-recruiting domains include, but are not limited to, GluR-2, GluR-B (R/G), GluR-B (Q/R), GluR-6 (R/G), 5HT2C, and FlnA (Q/R) domain (Aquino-Jarquin 2020). Typically, an ADAR-recruiting domain comprises a double-stranded RNA structure.

In one embodiment, the recruitment domain is capable of forming a stem-loop structure. Preferably, the recruitment domain has a nucleotide sequence as shown in SEQ ID NO: 1 to 4. More preferably, the recruitment domain has the nucleotide sequence as shown in SEQ ID NO: 1. The stem-loop structure may have at least two mismatches. The stem-loop structure may be formed from 10 to 100 nucleotides. Preferably, the stem-loop structure is formed from at least 20 to 80 nucleotides, 30 to 70 nucleotides or 40 to 60 nucleotides. More preferably, the stem-loop structure is formed from 45 to 49 nucleotides.

In another embodiment, the antisense oligonucleotide has a further element F linked to element A₁ at its 3′ end or linked to element A₃ at its 5′ end. It will be understood that element F may only be linked to element A3 when element E, i.e. the recruitment domain for recruiting ADAR, is not present. Preferably, element F is a nucleotide sequence capable of forming a secondary or tertiary structure. As used herein, a secondary structure refers to structures that are formed depending on how nucleotide bases form hydrogen bonds with each other. Tertiary structures refer to the three-dimensional shape of a nucleic acid polymer. Examples of secondary structures include, but are not limited to stem-loop structures (hairpin loop), pseudoknots and G-quadruplexes. Pseudoknots refer to double-hairpin structures with an extended quasi-continuous double-helical stem region. A pseudoknot is formed when bases outside a hairpin structure pair with bases within the hairpin or internal loop. It has significant roles in the biological function of RNA, such as ribosomal frameshifting. G-quadruplexes are four-stranded nucleic acid secondary structures formed by guanosine-rich DNA and RNA sequences. Preferably, the secondary structure is a hairpin loop or a pseudoknot structure and has a nucleotide sequence as shown in any one of SEQ ID Nos: 5 to 8. More preferably, the secondary structure is a pseudoknot structure with a nucleotide sequence as shown in SEQ ID NO: 5.

The present invention also relates to an expression cassette comprising a promoter sequence driving the expression of a nucleic acid encoding the antisense oligonucleotide as described herein.

The term “expression cassette” refers to a distinct component of vector DNA consisting of a gene and regulatory sequence allowing expression in prokaryotic or eukaryotic cells or isolated fractions thereof. For example, the antisense oligonucleotides may be ligated into a nucleic acid expression construct (i.e. vector) under the transcriptional control of a cis-regulatory sequence suitable for directing constitutive or inducible transcription of the nucleotide sequence in a cell. Typically, an expression cassette comprises a promoter sequence, an open reading frame and a 3′ untranslated region (3′ UTR). The untranslated region may also contain a polyadenylation site in order to increase efficiency of translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream.

The term “promoter” relates to a DNA sequence that initiates transcription of a single RNA transcript from the DNA downstream of the promoter. The term as used herein refers, thus, to DNA sequence elements that are required for initiation of transcription such as basal transcription factor binding sites required for the recruitment of RNA polymerases such as the TATA box. However, the term also encompasses enhancer binding sequences that are required to strengthen or facilitate expression. The person skilled in the art is well aware of which promoters may be used and how those can be used for expression of a gene of interest. In one embodiment, the promoter sequence is selected from the group consisting of: hU6-, H1- and h7SK-promoter. In another embodiment, the expression cassette further comprises a polyU terminator.

The antisense oligonucleotide may also be delivered to cells using a vector, such as viral vectors (e.g. lentiviral or adenoviral vectors from which they are then transcribed to produce the RNA antisense oligonucleotide). The antisense oligonucleotide may also be chemically produced, i.e. not relying on cell-based expression from plasmids or viruses. Preferably, the oligonucleotide of the invention is an RNA antisense oligonucleotide transcribed from a vector upon entry into the target cell.

Thus, the present invention also contemplates a vector comprising a nucleic acid encoding the antisense oligonucleotide or at least one expression cassette as described herein.

The term “vector” as used herein, refers to a polynucleotide that encodes a protein of interest. Preferably, the term “vector” encompasses phage, plasmid, cosmids, viral vectors as well as artificial chromosomes, such as bacterial or yeast artificial chromosomes (YAC). The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques. The terms “transformation” and “transfection”, conjugation and transduction, as used in the present context, are intended to comprise a multiplicity of prior-art processes for introducing foreign nucleic acid (for example DNA) into a host cell, including calcium phosphate, rubidium chloride or calcium chloride co-precipitation, DEAE-dextran-mediated transfection, lipofection, f-mating, natural competence, carbon-based clusters, chemically mediated transfer, electroporation or particle bombardment. Suitable methods for the transformation or transfection of host cells, including plant cells, can be found in standard text books such as Sambrook et al. (Molecular Cloning: A Laboratory Manual, 2nd ed., Cold Spring Harbor Laboratory, Cold Spring Harbor Laboratory Press, Cold Spring Harbor, NY, 1989). Alternatively, a plasmid vector may be introduced by heat shock or electroporation techniques. Should the vector be a virus, it may be packaged in vitro using an appropriate packaging cell line prior to application to host cells.

Preferably, the vector of the present invention is an expression vector. In such an expression vector, i.e. a vector which comprises the polynucleotide of the invention having the nucleic acid sequence operatively linked to an expression control sequence (also called “expression cassette”) allowing expression in prokaryotic or eukaryotic cells or isolated fractions thereof. Suitable expression vectors are known in the art such as pENTR (a kind gift of Dr. Joshua Rosenthal, University of Chicago), pLV (VectorBuilder), Okayama-Berg cDNA expression vector pcDV1 (Pharmacia), pCDM8, pRc/CMV, pcDNA1, pcDNA3 (Invitrogene) or pSPORT1 (GIBCO BRL). Further examples of typical fusion expression vectors are pGEX, pMAL (New England Biolabs, Beverly, MA) and pRIT5 (Pharmacia, Piscataway, NJ), where glutathione S transferase (GST), maltose E-binding protein and protein A, respectively, are fused with the recombinant target protein. Examples of suitable inducible non-fusion E. coli expression vectors are, inter alia, pTrc and pET 11d. The tar-get gene expression of the pTrc vector is based on the transcription from a hybrid trp-lac fusion promoter by host RNA polymerase. The target gene expression from the pET 11d vector is based on the transcription of a T7-gn10-lac fusion promoter, which is mediated by a co-expressed viral RNA polymerase (T7 gn1). This viral polymerase is provided by the host strains BL21 (DE3) or HMS174 (DE3) from a resident lambda-prophage which harbors a T7 gn1 gene under the transcriptional control of the lacUV 5 promoter. The skilled person is familiar with other vectors which are suitable in prokaryotic organisms; these vectors are, for example, in E. coli, pLG338, pACYC184, the pBR series such as pBR322, the pUC series such as pUC18 or pUC19, the M113mp series, pKC30, pRep4, pHS1, pHS2, pPLc236, pMBL24, pLG200, pUR290, pIN-III113-B1, lambdagt11 or pBdCl, in Streptomyces plJ101, plJ364, plJ702 or plJ361, in Bacillus pUB110, pC194 or pBD214, in Corynebacterium pSA77 or pAJ667. Examples of vectors for expression in the yeast S. cerevisiae comprise pYep Sec1, pMFa, pJRY88 and pYES2 (Invitrogen Corporation, San Diego, CA). Vectors and processes for the construction of vectors which are suitable for use in other fungi, such as the filamentous fungi, comprise those which are described in detail in standard text books such as van den Hondel, C. A. M. J. J., & Punt, P. J. (1991) “Gene transfer systems and vector development for filamentous fungi, in: Applied Molecular Genetics of fungi, J. F. Peberdy et al., Ed., pp. 1-28, Cambridge University Press: Cambridge, or in: More Gene Manipulations in Fungi (J. W. Bennett & L. L. Lasure, Ed., pp. 396-428: Academic Press: San Diego). Further suitable yeast vectors are, for example, pAG-1, YEp6, YEp13 or pEMBLYe23. As an alternative, the polynucleotides of the present invention can be also expressed in insect cells using baculovirus expression vectors. Baculovirus vectors which are available for the expression of proteins in cultured insect cells, e.g., Sf9 cells, comprise the pAc series and the pVL series.

In one embodiment, the vector according to the present invention comprises at least two expression cassettes, wherein the expression control sequences in at least two of the expression cassettes differ from each other. Expression control sequences as used herein refer to promotors, e.g. pU6-, H1-, h7SK-promoter and the like. Preferably, these expression control sequences differ from each if more than one expression cassette is present. For example, in a vector according to the present invention, one expression cassette may comprises the hU6 promoter and a second expression cassette may comprises the h7SK promoter. In certain examples, the vector may also comprises two expression cassettes, wherein the promoters do not differ from each other, e.g. both the first and the second expression cassette comprise the hU6 promoter.

The present invention further relates to the use of the antisense oligonucleotide, the expression cassette or the vector according to the invention for editing RNA in a cultured cell or in vitro. The editing of RNA will preferably take place in eukaryotic cells, preferably in metazoan cells, more preferably in animal cells or plant cells. Preferably, the RNA editing is used in mammalian cells, most preferably in human cells. Cells from any organ or tissue, e.g. skin, lung, heart, kidney, liver, pancreas, gut, muscle, gland, eye, brain, blood and the like can be used.

The cell targeted for RNA editing may have a genetic mutation. The mutation may be heterozygous or homozygous. For example, RNA editing as used herein could be used to modify point mutations, such as N to A mutations, wherein N may be G, C, or U/T, or N to C mutations, wherein N may be A, G, or U/T. However, it is also envisaged that the RNA editing is used in the opposite way, i.e. introducing a mutation into a cell, thus creating novel epitopes, also referred to as “neoepitopes”. A mutation to be modified through RNA editing may have arisen on the level of the chromosome or some other form of DNA, such as mitochondrial DNA, or RNA, including pre-mRNA, ribosomal RNA or mitochondrial RNA. A change to be made may be in a target RNA of a pathogen, including fungi, yeasts, parasites, kinetoplastids, bacteria, phages, viruses and the like, with which the cell or subject has been infected. Subsequently, the editing may take place on the RNA level on a target sequence inside such cell or pathogen.

The present invention also relates to a method for editing RNA comprising contacting the RNA to be edited in a cultured cell or in vitro with the antisense oligonucleotide according to the present invention.

The present invention further relates to a pharmaceutical composition comprising the antisense oligonucleotide, the expression cassette or the vector according to the present invention.

The term “pharmaceutical composition” as used herein, relates to compositions comprising the antisense oligonucleotide, the expression cassette or the vector of the present invention and, preferably, one or more pharmaceutically acceptable carrier. The pharmaceutical compositions are, preferably, administered systemically. Suitable routes of administration conventionally used for drug administration are oral, intravenous, subcutaneous, or parenteral administration as well as inhalation. However, depending on the nature and mode of action of a compound, the pharmaceutical compositions may be administered by other routes as well. Moreover, the antisense oligonucleotide, the expression cassette or the vector of the present invention can be administered in combination with other drugs either in a common pharmaceutical composition or as separated pharmaceutical composition, wherein said separated pharmaceutical compositions may be provided in form of a kit.

The pharmaceutical composition of the present invention is preferably administered in conventional dosage forms prepared by combining the drugs with standard pharmaceutical carriers according to conventional procedures. These procedures may involve mixing, granulating and compressing or dissolving the ingredients as appropriate for the desired preparation. It will be appreciated that the form and character of the pharmaceutically acceptable carrier or diluent is dictated by the amount of active ingredient with which it is to be combined, the route of administration and other well-known variables.

The carrier(s) must be acceptable in the sense of being compatible with the other ingredients of the formulation and being not deleterious to the recipient thereof. The pharmaceutical carrier employed may be, for example, a solid, a gel or a liquid. Exemplary of solid carriers are lactose, terra alba, sucrose, talc, gelatine, agar, pectin, acacia, magnesium stearate, stearic acid, degradable polymers like PLGA (DeYoung at al. (2011), DIABETES TECHNOLOGY & THERAPEUTICS 13:1145; Ramazani et al., (2016), Int J Pharm. 499(1-2): 358-367), and the like. Exemplary liquid carriers are phosphate buffered saline solution, syrup, oil such as peanut oil and olive oil, water, emulsions, various types of wetting agents, sterile solutions, and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pennsylvania.

The diluent(s) is/are selected so as not to affect the biological activity of the compound or compounds. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, non-immunogenic stabilizers, reactive oxygen scavengers, and the like.

The pharmaceutical composition is, preferably, administered together with standard pharmaceutical carriers according to conventional procedures. These procedures may involve mixing or dissolving the ingredients as appropriate to obtain the desired preparation. It will be appreciated that the form and character of the pharmaceutically acceptable carrier or diluent is dictated by the amount of antisense oligonucleotide, the expression cassette or the vector of the present invention with which it is to be combined, the route of administration and other well-known variables. Similarly, the carrier or diluent may include time delay material well known in the art, such as glyceryl mono-stearate, or glyceryl distearate alone or with a wax. A therapeutically effective dose refers to an amount of the antisense oligonucleotide, the expression cassette or the vector of the present invention to be used in a pharmaceutical composition of the present invention which provides the effect referred to in this specification. Therapeutic efficacy and toxicity of such compounds can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀. The dosage regimen will be determined by the attending physician and other clinical factors. As is well known in the medical arts, dosages for any one patient depend upon many factors, which may include the patient's size, body surface area, age, the particular compound to be administered, sex, time and route of administration, general health, and other drugs being administered concurrently. Progress can be monitored by periodic assessment. A typical dose can be, for example, in the range of 1 μg to 1000 mg; however, doses below or above this exemplary range are envisioned, especially considering the aforementioned factors.

The present invention also refers to an antisense oligonucleotide, an expression cassette or a vector according to the present invention for use in treating and/or preventing a disease or disorder associated with mutated RNA.

The term “treating” as used herein relates to ameliorating and/or curing diseases or disorders associated with mutated RNA, preventing progression of the disease and/or causing the reduction, remission or regression of a disease or disorder. Said treating as used herein also encompasses an entire restoration of health with respect to diseases or disorders associated with mutated RNA. It will be understood that a treatment as referred to herein will, in all likelihood, not be successful in all subjects which received the treatment. However, it is envisaged that the treatment is effective in at least a statistically significant portion of the subjects that are treated. Whether a statistically significant portion, e.g., of a cohort of subjects, can be successfully treated may, preferably, be determined, e.g., by statistical tests using various well known statistic evaluation tools, e.g., determination of confidence intervals, p-value determination, Student's t-test, Mann-Whitney test etc. Preferably, the treatment shall be effective for at least 10%, at least 20% at least 50% at least 60%, at least 70%, at least 80%, or at least 90% of the subjects of a given cohort or population.

The term “preventing” refers to retaining health with respect to diseases or disorders associated with mutated RNA for a certain period of time in a subject. It will be understood that the said period of time may be dependent on the therapy used, on the amount of the antisense oligonucleotide, the expression cassette or the vector of the present invention, which has been administered. It is to be understood that prevention may not be effective in all subjects that have been administered an antisense oligonucleotide, an expression cassette or a vector according to the present invention. However, the term requires that, preferably, a statistically significant portion of subjects of a cohort or population are effectively prevented from suffering from a disease or disorder associated with mutate RNA. Whether a portion is statistically significant can be determined without further ado by the person skilled in the art using various well-known statistic evaluation tools discussed elsewhere in this specification.

Preferably, said diseases or disorders associated with mutated RNA include cystic fibrosis, familial hypercholesterolaemia, Haemophilia-B, Tay-Sachs, ataxia telangiectasia, albinism, alpha-1-antitrypsin deficiency, Alzheimer disease, Amyotrophic lateral sclerosis, Asthma, ß-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophie Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Hurler Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonie dystrophy types 1 and II, neurofibromatosis, Niemann-Pick disease type A, Band C, NY-esol related cancer, Parkinson's disease, Peutz-Jeghers Syndrome, Phenylketonuria, 5 Pompe's disease, Primary Ciliary Disease, Prothrombin mutation related disorders, such as the Prothrombin G20210A mutation, Pulmonary Hypertension, Retinitis Pigmentosa, Sandhaff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, various forms of cancer (e.g. BRCAI and 2 linked breast cancer and ovarian 10 cancer), and the like.

Preferably, the disease or disorder associated with mutated RNA is cancer. Preferably, cancer that can be treated by the antisense oligonucleotide of the present invention include cancers that overexpress ADAR1. Cancers that over express ADAR1 include, but are not limited to, melanoma, liver cancer, esophageal, chronic myelogenous leukemia, ovarian cancer and breast cancer.

The present invention also relates to a method for treating and/or preventing a disease associated with mutated RNA comprising administering to a subject in need thereof a therapeutically effective amount of the antisense oligonucleotide, the expression cassette or the vector according to the present invention.

The term “subject” as used herein refers to an animal, preferably a vertebrate and, more preferably, a mammal. Thus, the herein provided means and methods are applicable to both human therapy and veterinary applications. Preferably, the mammal referred to herein is a pet, such as a dog, cat, or horse, or a farming animal, such as a cow, sheep, goat or pig, or a laboratory animal, such as a rodent and, preferably, a mouse or rat. Preferably, the mammal or laboratory animal referred to herein is a monkey. More preferably, the mammal referred to herein is a human.

A “therapeutically effective amount” refers to an amount of the antisense oligonucleotide of the invention which prevents or cures a disease or disorder associated with mutated RNA as referred to in this specification. Therapeutic efficacy and toxicity of the antisense oligonucleotide can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀. The dosage regimen will be determined by the attending physician and other clinical factors; preferably in accordance with any one of the above-described methods.

All explanations and definitions of the terms made above apply mutatis mutandis for the following embodiments.

The following embodiments are particular preferred embodiments according to the invention.

Embodiment 1: An antisense oligonucleotide having the following elements:

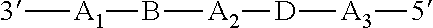

wherein
A₁ is a nucleotide sequence having a length of from 6 to 16 nucleotides;
B is the nucleotide cytosine;
A₂ is a nucleotide sequence having a length of from 33 to 37 nucleotides;
D is an internal loop-forming nucleotide sequence having a length of 4 nucleotides; and
A₃ is a nucleotide sequence having a length of from 10 to 20 nucleotides,
   wherein elements A₁, A₂ and A₃ are contiguous nucleotide sequences that are complementary to a target nucleotide sequence, and wherein the cytosine of element B forms a base-pair mismatch with adenosine in the target nucleotide sequence upon hybridization of the elements A₁, A₂ and A₃ to the target nucleotide sequence.

Embodiment 2: The antisense oligonucleotide of embodiment 1, wherein the antisense oligonucleotide is a single-stranded antisense oligonucleotide, preferably, a single-stranded antisense RNA oligonucleotide.

Embodiment 3: The antisense oligonucleotide of embodiment 1 or 2 having a further element E following 5′ of element A₃, wherein element E is a recruitment domain for recruiting a deaminase, preferably an ADAR1 recruiting domain or an ADAR2 recruiting domain.

Embodiment 4: The antisense oligonucleotide of embodiment 3, wherein the recruitment domain is capable of forming a stem loop structure and has a nucleotide sequence as shown in SEQ ID No: 1 to 4.

Embodiment 5: The antisense oligonucleotide of any one of embodiments 1 to 4 having a further element F linked to element A₁ at its 3′ end or linked to element A₃ at its 5′ end when element E is not present, wherein element F is a nucleotide sequence capable of forming a secondary or tertiary structure.

Embodiment 6: The antisense oligonucleotide of embodiment 5, wherein the secondary structure is a hairpin loop or a pseudoknot structure and has a nucleotide sequence as shown in any one of SEQ ID Nos: 5 to 8.

Embodiment 7: An expression cassette comprising a promoter sequence driving the expression of a nucleic acid encoding the antisense oligonucleotide of any one of embodiments 1 to 6.

Embodiment 8: The expression cassette of embodiment 7, wherein the promoter sequence is selected from the group consisting of: hU6-, H1-, and h7SK-promoter.

Embodiment 9: The expression cassette of embodiment 7 or 8, wherein the expression cassette further comprises a polyU terminator.

Embodiment 10: A vector comprising a nucleic acid encoding the antisense oligonucleotide of any one of embodiments 1 to 6 or at least one expression cassette of any one of embodiments 7 to 9.

Embodiment 11: The vector of embodiment 10, wherein said vector comprises at least two expression cassettes according to any one of embodiments 7 to 9, wherein the expression control sequences in at least two of the expression cassettes differ from each other.

Embodiment 12: Use of the antisense oligonucleotide of any one of claims 1 to 6, the expression cassette of any one of embodiments 7 to 9 or the vector of embodiment 10 or 11 for editing RNA in a cultured cell or in vitro.

Embodiment 13: A method for editing RNA comprising contacting the RNA to be edited in cultured cell or in vitro with the antisense oligonucleotide of any one of embodiments 1 to 6.

Embodiment 14: A pharmaceutical composition comprising the antisense oligonucleotide of any one of embodiments 1 to 6, the expression cassette of any one of embodiments 7 to 9 or the vector of any one of embodiments 10 or 11.

Embodiment 15: An antisense oligonucleotide of any one of embodiments 1 to 6, an expression cassette of any one of embodiments 7 to 9 or a vector of embodiment 10 or 11 for use in treating and/or preventing a disease or disorder associated with mutated RNA.

Embodiment 16: The antisense oligonucleotide, the expression cassette or the vector for use of embodiment 15, wherein the disease associated with mutated RNA is cancer.

Embodiment 17: A method for treating and/or preventing a disease associated with mutated RNA comprising administering to a subject in need thereof a therapeutically effective amount of the antisense oligonucleotide of any one of claims 1 to 6, the expression cassette of any one of embodiments 7 to 9 or the vector of any one of embodiments 10 or 11.

Embodiment 18: The method of embodiment 17, wherein the disease associated with mutated RNA is cancer.

All references cited throughout the specification are herewith incorporated by reference with respect to the specifically mentioned disclosure content as well as in their entireties.

### FIGURES

FIG. 1: Flow-cytometry analysis to evaluate ASO-dependent RNA editing. (A) Schematic representation of the reporter cell line used in this study. The ASO binds the mcherry-eGFP mRNA, creates a dsRNA, and recruits ADAR1. ADAR1 editing leads to the activation of eGFP, and the efficiency of this process can be measured via flow cytometry by counting the number of cells that are mCherry⁺-eGFP⁺. (B) Direct comparison between 3 previously published ASOs. On the left is a histogram showing the results of flow-cytometry analysis, and on the right a schemate showing the structure of the three ASOs. (C) Optimization of ASO number 3, represented in a general form on the top of the panel. The two histograms (bottom left and right) show changes in editing efficiency (measured as % of GFP+ cells) by reducing the length of the ASO's 3,′ and 5′ ends. For each bar plot: center=mean and error bars=standard deviation, n=3.

FIG. 2: The position of the internal loop-forming nucleotide sequence affects editing efficiency. (A) Schematic representative the ASOs used in this experiment. (B) Bar plot showing changes in editing efficiency (measured as % of GFP+ cells) by increasing the length of fragment A2 and therefore the position of internal loop-forming nucleotide sequence. For each bar plot: center=mean and error bars=standard deviation, n=3.

FIG. 3: The shorter ASO is more efficient and precise. (A) Flowchart of the experiment. (B) Schematic representation of the general structure of the ASOs tested in C and D. (C) Bar plot of the on-target editing efficiency measured by Sanger sequencing for each ASO. Center=mean and error bars=standard deviation, n=3. (D) Quantification of editing for each ASO based on sequences from bacterial colonies. Each dot on the x-axis is an A within the eGFP-sequenced amplicon (n=20).

FIG. 4: Expression and stability of the ASOs affect their editing efficiency. (A) Schematic representation of the ASO used in B-D. (B) Bar plot showing the editing efficiency using the same ASO but under the control of three different polIII promoters (upper part shows a schemate of the plasmids used). (C) Bar plot showing the editing efficiency using an increasing number of cassettes per plasmid. In the first four samples from the left, only hU6 was used as promoters, while in the last three samples, each plasmid contains cassettes whose expression is driven by hU6 or h7SK. (D) Bar plot showing the editing efficiency of ASO with different RNA structures at its 3′end. histone 3′UTR mutated stem-loop=SLmt; histone 3′UTR stem-loop (SL); truncated and modified version of prequeosine1-1 riboswitch aptamer (teQ1); pseudoknot from Moloney murine leukemia virus (tmp). For each bar plot: center=mean and error bars=standard deviation, n=3.

### SEQUENCES

The following sequences are referred to in the application:

SEQ ID NO: 1: Nucleic acid sequence of DDX58 loop 48 nt v1.

SEQ ID NO: 2: Nucleic acid sequence of DDX58 loop 48 nt v2.

SEQ ID NO: 3: Nucleic acid sequence of GluR loop v1.

SEQ ID NO: 4: Nucleic acid sequence of GluR loop v9.4.

SEQ ID NO: 5: Nucleic acid sequence of histone 3′UTR mutated stem-loop (SLmt).

SEQ ID NO: 6: Nucleic acid sequence of histone 3′UTR stem-loop (SL).

SEQ ID NO: 7: Nucleic acid sequence of a truncated and modified version of prequeosine₁-1 riboswitch aptamer (teQ1).

SEQ ID NO: 8: Nucleic acid sequence of the frameshifting pseudoknot from Moloney murine leukemia virus (tmp).

### EXAMPLES

The Examples shall merely illustrate the invention. They shall, whatsoever, not be construed as limiting the scope.

### Materials and Methods

### Cell Lines

HEK293T cells (obtained from DKFZ, ATCC, Cat #CRL-3216, RRID: CVCL_0063) were cultured at 37° C., 5% CO₂ in high-glucose DMEM (Sigma-Aldrich, Cat #D6429) supplemented with 10% FBS (PAN Biotech, Cat #P40-37100) and 1% penicillin/streptomycin (Sigma-Aldrich, Cat #P4333). The cell line was authenticated using Multiplex Cell Authentication by Multiplexion (Heidelberg, Germany). Additionally, the purity of the cell line was validated using the Multiplex cell Contamination Test by Multiplexion (Heidelberg, Germany). No Mycoplasma, SMRV, or interspecies contamination was detected.

### Plasmids

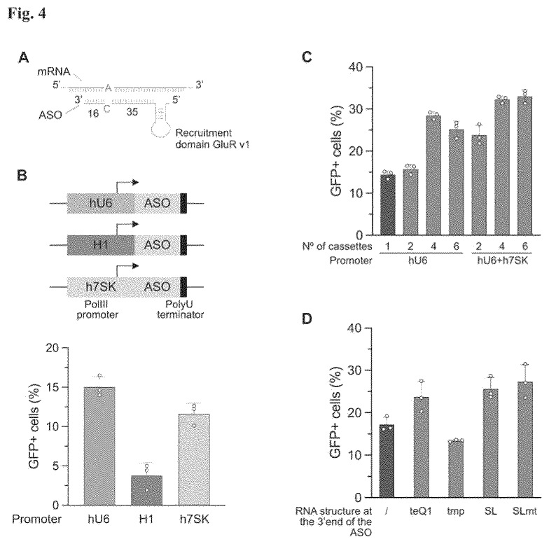

mCherry-T2A-eGFP W58X and U6 pENTR gRNA vectors were a kind gift of Dr. Joshua Rosenthal (University of Chicago) (Montiel-González et al. 2016). The mCherry-T2A-eGFP W58X was modified by inserting a puromycin resistance cassette within the BglII restriction site. All the ASOs coding plasmids were generated using the U6 pENTR gRNA vector as the backbone in which the gRNA was substituted with different ASOs. When different promoters were used, the U6 sequence was replaced with the sequence of other promoters (H1 or h7SK) (FIG. 4).

### Example 1: Generation of a HEK293T mCherry-T2A-eGFP W58X Reporter Cell Line and Transfections with Plasmids Coding ASOs

HEK293T cells were seeded in 24-well plates (~150,000 cells per well) to have a confluency of 70-90% the following day. After 24 h, cells were transfected with 2 g of the mCherry-T2A-eGFP W58X reporter plasmid using Lipofectamine 2000. Then, 48 h after transfections, cells were diluted in 96-well plates and selected using puromycin (1.5 g/ml) for two weeks. Clonality was validated by visual inspection with a microscope, and the clones were then screened for the presence of mCherry via flow-cytometry analysis.

This cell line was then transfected to assess the efficiency of ASOs in recruiting endogenous ADARs. HEK293T mCherry-T2A-eGFP W58X and cells were seeded in 24-well plates (~150,000 cells per well). After 24 h, cells were transfected with 1.5 g of ASO coding plasmid using Lipofectamine 2000. The efficiency of ADARs recruitment was measured at 24 h, 48 h, and 72 h via flow-cytometry analysis using mCherry⁺-eGFP⁺ cells.

### Example 2

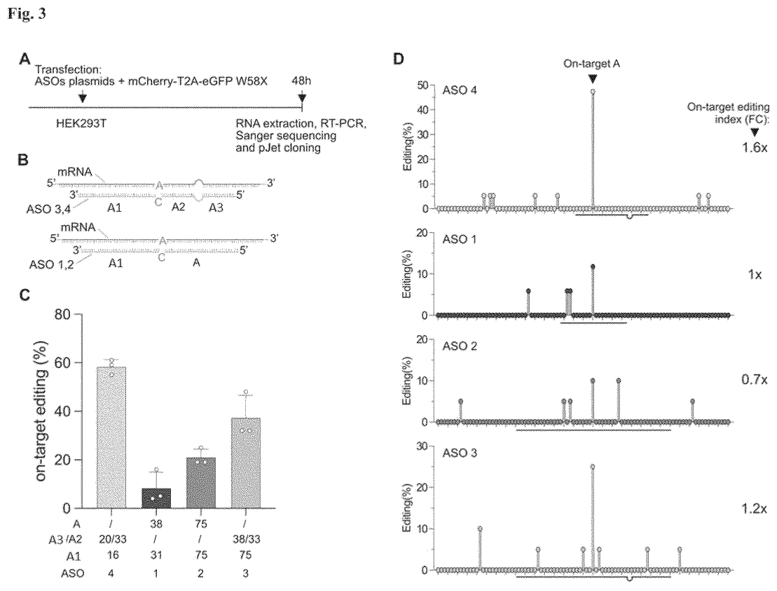

### On-Target and Off-Target Analysis of ASO-Dependent RNA Editing (FIG. 3)

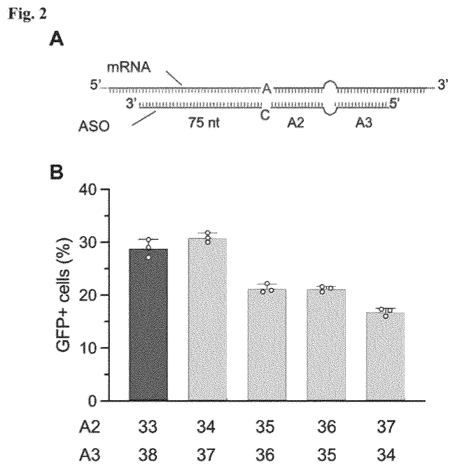

To evaluate on-target and off-target ASO-dependent editing on eGFP, HEK293T cells were transfected with 25 ng of mCherry-T2A-eGFP W58X reporter plasmid and 1.5 g of ASO coding plasmid using Lipofectamine 2000. 48 h after transfection, RNA was extracted using the RNeasy Plus Mini kit (Qiagen, Cat #74134) and treated with DNase (Invitrogen, Cat #AM1907). Following RNA extraction, RT-PCRs were performed with gene-specific primers (Forward: AACTTCAGCCTGCTCAAACAAGCC; Reverse: CAGCCCTGGTCTTGTAGTTG) and a One-step RT-PCR kit (Qiagen, Cat #210212). PCR products were gel extracted, purified (Macherey-Nagel, Cat #740609), and analyzed by Sanger sequencing. Quantification of editing was performed directly from the Sanger traces using MultiEditR (Kluesner et al. 2021), as shown in FIG. 2C. Alternatively, the PCR products were cloned using a CloneJET PCR cloning kit (Thermo Scientific, Cat #K1232) according to the manufacturer's instructions and transformed into DH5a bacteria (NEB, Cat #C2987). Ten to fifty resultant bacteria colonies were sent for sequencing to determine edits and their frequency in the targeted region, as shown in FIG. 2D. To evaluate the precision of each ASO, the Amplicon Editing Index (AmEI) was calculated as the ratio between the “number of edited As” and “the total number of As” in the amplicon. AmEI thus measures the total amount of editing (on-target and off-target) in each amplicon. Finally, to evaluate the precision of each ASO, an on-target editing index was generated as the ratio between “number of on-target edited As” and AmEI. This index taking into consideration both the on-target and off-target editing allow quantitative evaluation of the ASO precision.

### Results

The Presence of an Internal Loop-Forming Sequence within the ASO Improves Editing.

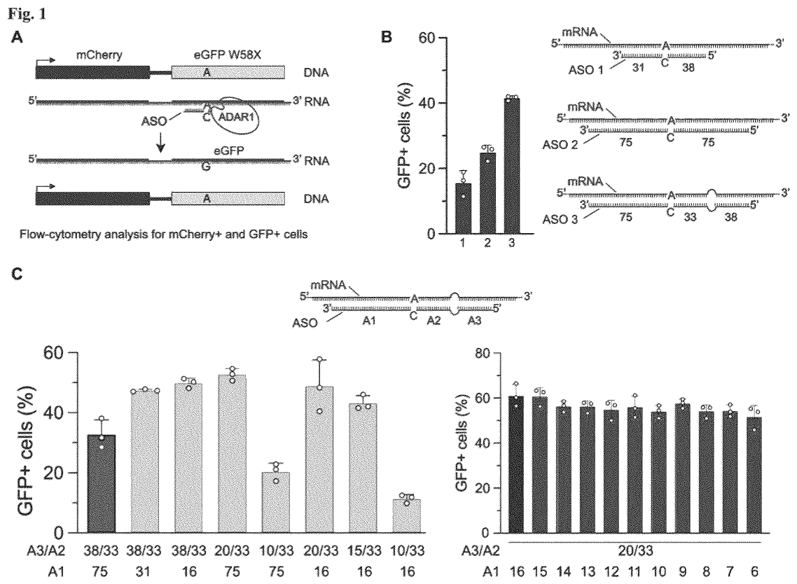

The capability of plasmid-encoded ASOs to recruit endogenous ADARs was tested in a HEK293T reporter cell line stably expressing mCherry and an inactive eGFP mutant in which a tryptophan is mutated in a stop codon (W58X). This mutation of eGFP can be reverted to the wild-type version through ASO-dependent ADAR editing, and eGFP intensity enables quantification of the efficiency of RNA editing. Additionally, the presence of mCherry allows to check for possible and undesired RNA interference effects on the transcript due to ASO binding (FIG. 1A). Using this system, in the first experiment, three different previously published ASOs to their capability to recruit endogenous ADAR (Qu et al 2019; Uzonyi et al 2021) were compared (FIG. 1i). ASOs 1 and 2 are perfectly complementary to the eGFP mRNA target, except for an A:C mismatch, which defines the A to be targeted. Differently, ASO 3 contains another four nt mismatches centered in position −35 compared to the A:C mismatch, generating an internal loop structure between the mRNA target and the ASO. Finally, ASO 1 length is 70 nt, while ASO 2 and 3 are 151 nt long (FIG. 1B, right side). ASO 3 was the most efficient in recruiting endogenous ADAR, leading to the highest number of GFP+ cells (FIG. 1B, left side). Considering that the only difference between ASO 2 and 3 is the presence of the internal loop-forming sequence, these results demonstrate how crucial this feature is to efficiently recruit ADAR in an ASO-dependent manner.

Considering that a long ASO will also lead to unwanted off-target editing on the mRNA target, it was tested whether the length of the ASO could be further reduced without a decrease in editing efficiency. Notably, it was possible to reduce the size of the ASO to 74 nt, shortening both the 5′ and 3′ ends of the ASO while obtaining an increase in editing efficiency (FIG. 1C, left side). Shortening the 5′ end of the ASO resulted in an increase of editing when the segment A1 was equivalent to 20 or 15 nt, while a shortening to 10 nt lead to a ~3-fold decrease in editing efficiency (FIG. 1C, left side). Therefore, a 5′end length of 20 nt was used. Additionally, we have also tested whether the length of fragment A2 of the ASO affects editing efficiency. Lengths of 33 or 34 nt lead to the higher editing efficiencies and therefore a length of 33 nt was used (FIG. 2).

Finally, it was tested whether the length of the 3′end of the ASO (from 16 to 6 nt) could be further decreased. However, a decrease in the percentage of the GFP+ cells up to 10% for ASO shorter than 16 nt (FIG. 1C) was observed, and therefore a 3′end length of 16 nt was used. Finally, the resulting optimized ASO was called ASO 4.

### The Short ASO is More Efficient and Precise.

To quantify the efficiency and the precision of the optimized short ASO (ASO 4), the first step is to evaluate the editing efficiency of the targeted base (on-target editing), and the second step is to assess the presence of possible undesired editing sites along the same transcript (off-target editing). With this goal in mind, HEK293T cells were transfected with the mCherry-T2A-eGFP W58X reporter plasmid and plasmids coding ASO 1-4. 48 h after transfection following RNA extraction, RT-PCR was performed on eGFP, and the amplicons were Sanger sequenced. On-target editing was evaluated directly from Sanger traces, while to evaluate off-target editing, PCR products were first introduced into bacteria, and then single bacterial colonies were sequenced using Sanger sequencing. Alignment to the unedited reference plasmid allowed to easily count the editing sites in the presence of the different ASOs to assess their on-target and off-target editing efficiency. ASO 4 resulted in the highest on-target editing measured directly from Sanger sequencing (FIG. 3C) and bacterial colonies (FIG. 3D). Notably, ASO 4 also results in the lowest level of off-target editing as measured by the highest on-target editing index value, with a 1.6-fold increase compared to ASO 1 (FIG. 3D).

### ASOs Expression and Stability Affect Editing Efficiency.

The steady-state level of any RNA in a cell at a certain moment is dictated by its synthesis and decay rates. Based on this principle, it was tested whether changes in the expression level or stability of an ASO could affect its ability to recruit ADAR and, therefore, its editing efficiency. To test this aspect, an ASO developed in our lab was used, composed of a short specificity domain (the region complementary to the mRNA target) and a recruitment domain at the 5′ end of the ASO (FIG. 4A). This ASO is an optimized version of the RESTORE design recently published (Merkle et al. 2019) but without chemical modifications and encoded in a plasmid. Different polIII promoters to drive ASO expression were tested first. Despite hU6 resulting in the highest editing, the h7SK promoter also led to a good level of editing (FIG. 4B). To achieve higher levels of ASO expression, the number of cassettes for ASO expression present in a single plasmid was increased. Up to six cassettes per plasmid, in which the expression was driven only by hU6 or by hU6 and h7SK, were tested. Interestingly, the best editing was obtained in the presence of 4 or 6 cassettes per plasmid, with half of the cassettes containing hU6 and the other half h7SK.

Finally, a recent paper has shown that RNA structures at the 3′end of gRNA for Cas9 prime editing led to a decrease in the degradation of gRNAs followed by an increase in DNA editing (Nelson et al. 2022). Based on these findings, it was tested whether the presence of RNA structures at the 3′end of the ASO would increase editing efficiency. Three out of four RNA structures increased the ASO-dependent editing efficiency, with the highest editing achieved by a mutated version of the histone 3′UTR stem-loop motif (FIG. 4D).

### CITED LITERATURE

Battle D J, Doudna J A. “The stem-loop binding protein forms a highly stable and specific complex with the 3′ stem-loop of histone mRNAs”. RNA. 2001 January; 7(1):123-32. doi: 10.1017/s1355838201001820
Merkle T, Merz S, Reautschnig P et al. “Precise RNA editing by recruiting endogenous ADARs with antisense oligonucleotides”. Nat Biotechnol 37, 133-138 (2019). https://doi.org/10.1038/s41587-019-0013-6
Qu L, Yi Z, Zhu S et al. “Programmable RNA editing by recruiting endogenous ADAR using engineered RNAs”. Nat Biotechnol 37, 1059-1069 (2019). https://doi.org/10.1038/s41587-019-0178-z
Uzonyi A, Nir R, Shliefer P et al. “Deciphering the principles of the RNA editing code via large-scale systematic probing”. Molecular Cell, vol. 81, issue 11, 2374-2387 (2021). https://doi.org/10.1016/j.molcel.2021.03.024
WO 2022/091100 A1
Kluesner M G, Tasakis R N, Lerner T et al. “MultiEditR: The first tool for the detection and quantification of RNA editing from Sanger sequencing demonstrates comparable fidelity to RNA-seq”. Molecular Therapy Nucleic Acids (2021), vol. 25, DOI: https://doi.org/10.1016/j.omtn.2021.07.008
Montiel-González M F, Vallecillo-Viejo I C, Rosenthal J J. “An efficient system for selectively altering genetic information within mRNAs”. Nucleic Acids Res. 2016 Dec. 1; 44(21):e157. doi: 10.1093/nar/gkw738
Hirao I, Kimoto M. “Unnatural base pair systems toward the expansion of the genetic alphabet in the central dogma”. Proc Jpn Acad Ser B Phys Biol Sci. 2012 Jul. 25; 88(7): 345-367. doi: 10.2183/pjab.88.345
Aquino-Jarquin G. “Novel Engineered Programmable Systems for ADAR-Mediated RNA Editing”. Molecular Therapy Nucleic Acids, vol. 19, DOI:https://doi.org/10.1016/j.omtn. 2019.12.042

1. An antisense oligonucleotide having the following elements: 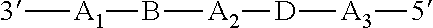
wherein
A₁ is a nucleotide sequence having a length of from 6 to 16 nucleotides;
B is the nucleotide cytosine;
A₂ is a nucleotide sequence having a length of from 33 to 37 nucleotides;
D is an internal loop-forming nucleotide sequence having a length of 4 nucleotides; and
A₃ is a nucleotide sequence having a length of from 10 to 20 nucleotides,
wherein elements A₁, A₂ and A₃ are contiguous nucleotide sequences that are complementary to a target nucleotide sequence, and wherein the cytosine of element B forms a base-pair mismatch with adenosine in the target nucleotide sequence upon hybridization of the elements A₁, A₂ and A₃ to the target nucleotide sequence.

2. The antisense oligonucleotide of claim 1, wherein the antisense oligonucleotide is a single-stranded antisense oligonucleotide, preferably, a single-stranded antisense RNA oligonucleotide.

3. The antisense oligonucleotide of claim 1 having a further element E following 5′ of element A₃, wherein element E is a recruitment domain for recruiting a deaminase, preferably an ADAR1 recruiting domain or an ADAR2 recruiting domain.

4. The antisense oligonucleotide of claim 3, wherein the recruitment domain is capable of forming a stem loop structure and has a nucleotide sequence as shown in SEQ ID No: 1-4.

5. The antisense oligonucleotide of claim 1 having a further element F linked to element A1 at its 3′ end or linked to element A3 at its 5′ end when element E is not present, wherein element F is a nucleotide sequence capable of forming a secondary or tertiary structure.

6. The antisense oligonucleotide of claim 5, wherein the secondary structure is a hairpin loop or a pseudoknot structure and has a nucleotide sequence as shown in any one of SEQ ID Nos: 5 to 8.

7. An expression cassette comprising a promoter sequence driving the expression of a nucleic acid encoding the antisense oligonucleotide of claim 1.

8. The expression cassette of claim 7, wherein the promoter sequence is selected from the group consisting of: hU6-, H1-, and h7SK-promoter.

9. The expression cassette of claim 7, wherein the expression cassette further comprises a polyU terminator.

10. A vector comprising a nucleic acid encoding the antisense oligonucleotide of claim 1 or at least one expression cassette comprising a promoter sequence driving the expression of a nucleic acid encoding the antisense oligonucleotide of claim 1.

11. A vector comprising at least two expression cassettes, wherein the expression cassettes each comprise a promoter sequence driving the expression of a nucleic acid encoding the antisense oligonucleotide of claim 1, and wherein the expression control sequences in at least two of the expression cassettes differ from each other.

12. (canceled)

13. A method for editing RNA comprising contacting the RNA to be edited in cultured cell or in vitro with the antisense oligonucleotide of claim 1.

14. A pharmaceutical composition comprising the antisense oligonucleotide of claim 1.

15. (canceled)

16. A method for treating and/or preventing a disease associated with mutated RNA, preferably cancer, comprising administering to a subject in need thereof a therapeutically effective amount of the antisense oligonucleotide of claim 1.
